(19) United States
(12) Patent Application Publication
(10) Pub. No.: US 2022/0401688 A1
(43) Pub. Date: Dec. 22, 2022

(54) SYSTEMS AND METHODS FOR ENHANCED OXYGEN DELIVERY

(71) Applicant: Covidien LP, Mansfield, MA (US)

(72) Inventors: Ryan A. Gleim, Vista, CA (US); Nicole M. Gibson, San Diego, CA (US); Kristen A. Hood, Sachse, CA (US)

(73) Assignee: Covidien LP, Mansfield, MA (US)

(21) Appl. No.: 17/806,104

(22) Filed: Jun. 9, 2022

Related U.S. Application Data
(60) Provisional application No. 63/211,289, filed on Jun. 16, 2021.

(51) Int. Cl. A61M 16/20; G06F 3/04847; A61M 16/00; A61M 16/10

(52) U.S. Cl. CPC A61M 16/202; G06F 3/04847; A61M 16/024; A61M 16/1005; A61M 2205/502; A61M 2230/43

(57) ABSTRACT

A method of controlling a ventilation system includes displaying, on a display screen of a ventilation system, a first bar including a plurality of parameter segments. Each parameter segment indicates a level of a ventilation setting. The method further includes displaying, on the display screen, a second bar including a plurality of time segments. Each time segment indicates a time duration of a corresponding parameter segment of the first bar. As time passes, the display of the second bar is altered based on an amount of time passing. Upon a time duration of a time segment expiring, the ventilation setting is altered by the ventilation system to a level corresponding to a subsequent parameter segment. The display of the first bar is altered to highlight the subsequent parameter setting.

### CROSS-REFERENCE TO RELATED APPLICATIONS

This application claims the benefit of U.S. Provisional Application No. 63/211,289 filed Jun. 16, 2021, entitled “Systems and Methods for Enhanced Oxygen Delivery,” which is incorporated herein by reference in its entirety.

### INTRODUCTION

Medical ventilator systems have long been used to provide ventilatory and supplemental oxygen support to patients. These ventilators typically comprise a connection for pressurized gas (air, oxygen) that is delivered to the patient through a conduit or tubing. As each patient may require a different ventilation strategy, modern ventilators may be customized for the particular needs of an individual patient. For example, a patient may need oxygen levels that differ from standard delivery levels or a temporary adjustment of oxygen levels.

It is with respect to these and other general considerations that the aspects disclosed herein have been made. Also, although relatively specific problems may be discussed, it should be understood that the examples should not be limited to solving the specific problems identified in the background or elsewhere in this disclosure.

### SUMMARY

In one aspect, the technology relates to a method of controlling a ventilation system, the method comprising: displaying, on a display screen of a ventilation system, a first bar including a plurality of parameter segments, each parameter segment indicating a level of a ventilation setting; displaying, on the display screen, a second bar including a plurality of time segments, each time segment indicating a time duration of a corresponding parameter segment of the first bar; as time passes, altering the display of the second bar based on an amount of time passing; upon a time duration of a time segment expiring: altering, by the ventilation system, the ventilation setting to a level corresponding to a subsequent parameter segment; and altering the display of the first bar to highlight the subsequent parameter setting. In an example, the first bar and the second bar are displayed as concentric, adjacent rings. In another example, the method further comprises displaying a current level of the ventilation setting within the rings formed by the first bar and the second bar. In yet another example, the method further comprises displaying a selectable user interface element for controlling at least one of: activation of a boost mode, pausing of the boost mode, or stopping a boost mode.

In another example of the above aspect, the ventilation setting is a fraction of inspired oxygen (FiO2) setting. In another example, the ventilation setting is a positive end-expiratory pressure (PEEP). In yet another example, each parameter segment displays a number corresponding to the level of the ventilation setting. In still another example, the parameter segments of the first bar are aligned with the time segments of the second bar.

In another aspect, the technology relates to a ventilation system comprising: a display screen; and a controller including at least one processor and memory storing instructions that, when executed by the at least one processor cause the system to perform operations comprising: displaying, on a display screen of a ventilation system, a first bar including a plurality of parameter segments, each parameter segment indicating a level of a ventilation setting; displaying, on the display screen, a second bar including a plurality of time segments, each time segment indicating a time duration of a corresponding parameter segment of the first bar; as time passes, altering the display of the first bar based on an amount of time passing; upon a time duration of a time segment expiring: altering, by the ventilation system, the ventilation setting to a level corresponding to a subsequent parameter segment; and altering the display of the first bar to highlight the subsequent parameter setting. In an example, the display screen is attached to a ventilator housing. In another example, the ventilation setting is a fraction of inspired oxygen (FiO2) setting or a PEEP setting. In yet another example, the system further comprises an oxygen valve, and altering the ventilation setting includes adjusting the oxygen valve.

In another example of the above aspect, the controller, when executed by the at least one processor causes the system to perform operations further comprising: receiving, from a user interface, a selection of a final ventilation level, a selection of a total time duration, and a selection of a number of time segments. In an example, the duration of time of each of the plurality of time segments is selected by movement of a selector on a user interface. In yet another example, the level of the ventilation setting at each parameter segment is selected based on input received from a user interface.

In another aspect, the technology relates to a method for controlling a ventilator, the method comprising: delivering ventilation at a first FiO2 level; receiving an input to activate an oxygen boost process; displaying, on a display screen of a ventilation system, a first bar including a plurality of parameter segments, each parameter segment indicating an FiO2 level; displaying, on the display screen, a second bar including a plurality of time segments aligned with corresponding parameter segments of the first bar, each time segment indicating a time duration of a corresponding parameter segment of the first bar; in response to receiving the input to activate the oxygen boost process: delivering ventilation at second FiO2 level corresponding to a first parameter segment of the first bar, the second FiO2 level being greater than the first FiO2 level; and highlighting the first parameter segment; upon expiration of a first time duration corresponding to a first time segment of the second bar: delivering ventilation at a third FiO2 level corresponding to a second parameter segment of the second bar; and highlighting the second parameter segment. In an example, the third FiO2 level is less than the second FiO2 level and greater than the first FiO2 level. In another example, the third FiO2 level is greater than the second FiO2 level. In yet another example, the method further comprises, upon expiration of a second time duration corresponding to a second time segment of the second bar: delivering ventilation at a fourth FiO2 level corresponding to a third parameter segment of the second bar, the fourth FiO2 level being less than the third FiO2 level and greater than the first FiO2 level; and highlighting the third parameter segment. In still another example, the method further comprises: in response to one of receiving an input to stop the oxygen boost process or expiration of a time duration of a final segment of the second bar: delivering ventilation at the first FiO2 level.

### BRIEF DESCRIPTION OF THE DRAWINGS

The following drawing figures, which form a part of this application, are illustrative of aspects of systems and methods described below and are not meant to limit the scope of the disclosure in any manner, which scope shall be based on the claims.

FIG. 1 is a diagram illustrating an example of a medical ventilator connected to a human patient.

FIG. 2 is an example display screen having a graphical user interface coupled to a ventilator.

FIGS. 3A and 3B are further example display screens having graphical user interfaces coupled to a ventilator.

FIG. 4 is another example display screen having a graphical user interface coupled to a ventilator.

FIG. 5 is another example display screen having a graphical user interface with selectable oxygenation settings coupled to a ventilator.

FIG. 6 depicts an example method of controlling a temporary oxygenation adjustment function of a ventilation system.

FIG. 7 depicts another example method of controlling a temporary oxygenation adjustment function of a ventilation system.

While examples of the disclosure are amenable to various modifications specific aspects have been shown by way of example in the drawings and are described in detail below. The intention is not to limit the scope of the disclosure to the particular aspects described. On the contrary, the disclosure is intended to cover all modifications, equivalents, and alternatives falling within the scope of the disclosure and the appended claims.

### DETAILED DESCRIPTION

As discussed briefly above, medical ventilators are used to provide breathing gases to patients who are otherwise unable to breathe sufficiently. The mode of ventilation and/or the settings for ventilation may be set based on the particular patient and any clinical conditions of the patient, such as asthma, acute respiratory distress syndrome (ARDS), emphysema, chronic obstructive pulmonary disease (COPD), or bronchopulmonary dysplasia (BPD) among others. By properly adjusting the ventilator settings based on the clinical condition of the patient, the ventilator can better support the patient, and the patient may be more likely to recover quickly or be weaned from the ventilator more quickly. In some cases, a patient may require a temporary adjustment in oxygen levels, such as an increase in delivered oxygen level or a reduction in oxygen level. In traditional systems, such an adjustment of oxygen was done manually, which created the inherent risk that a clinician may get distracted and not readjust the oxygen level back to the baseline level. Leaving the oxygen level at the increased level for an unintended long period of time can potentially cause severe injuries to the patient, including lung damage or even blindness in neonatal patients.

One option to prevent the oxygen level from remaining at an increased level for an undesired length of time is to use a timer that automatically causes the oxygenation level to revert to the normal or baseline level at the expiration of the timer. Such an approach, however, can cause a dramatic increase and decrease in oxygenation levels, which can be physiologically inappropriate for the patient. For instance, a patient may have a baseline oxygenation level of 40%, and the temporarily adjusted oxygenation level is 100%. Abruptly increasing the oxygenation level from 40% to 100% and then back from 100% to 40% can be physiologically inappropriate for the patient as slower transition allow adequate compensation periods.

The present technology improves such ventilation technology by using a tiered approach to adjusting the oxygenation levels for temporary adjustments. Using a tiered approach to increase or decrease oxygen levels over time with several tiers in between the initial oxygen level and the final oxygen level, with each tier having a different oxygen level, puts less stress on a patient, as opposed to making abrupt changes in oxygen levels. Furthermore, each tier may have an associated timer. The timers associated with each of the tiers helps prevent over and/or under oxygenation by adjusting oxygen levels automatically when a timer expires. For example, once a timer for a respective tier has expired, the oxygen level being administered to the patient may be adjusted to the next tier until all tiers have been completed. Once all tiers have been completed, the ventilator may return to the baseline oxygen level that was being delivered prior to initiation of the temporary enhanced oxygenation function. This helps ensure patient safety and avoids the risk of over-oxygenating a patient, even if a clinician must briefly step away from the patient while oxygen levels are being temporarily boosted.

The progression of the tiers is displayed on a unique graphical user interface (GUI) that may be displayed on a display screen of the ventilator. The GUI may have selectable user interface elements for temporarily adjusting the oxygenation levels and paired time durations of the ventilation system. The selectable user interface elements enable a clinician to easily configure a cycle for temporarily adjusting the oxygenation levels of a patient. For example, a clinician can input the number of tiers or segments in the cycle, the paired oxygenation levels, and the time durations of the level or cycle. Once the oxygenation settings are configured, the display screen may show the segments of the cycle in a concentric ring. Over time as the cycle progresses, the display of segments that have been completed may change (e.g., completed segments may be greyed out). The display screen and selectable user interface provide efficiency and clarity in configuring and enabling a temporary oxygenation adjustment function associated with a ventilator.

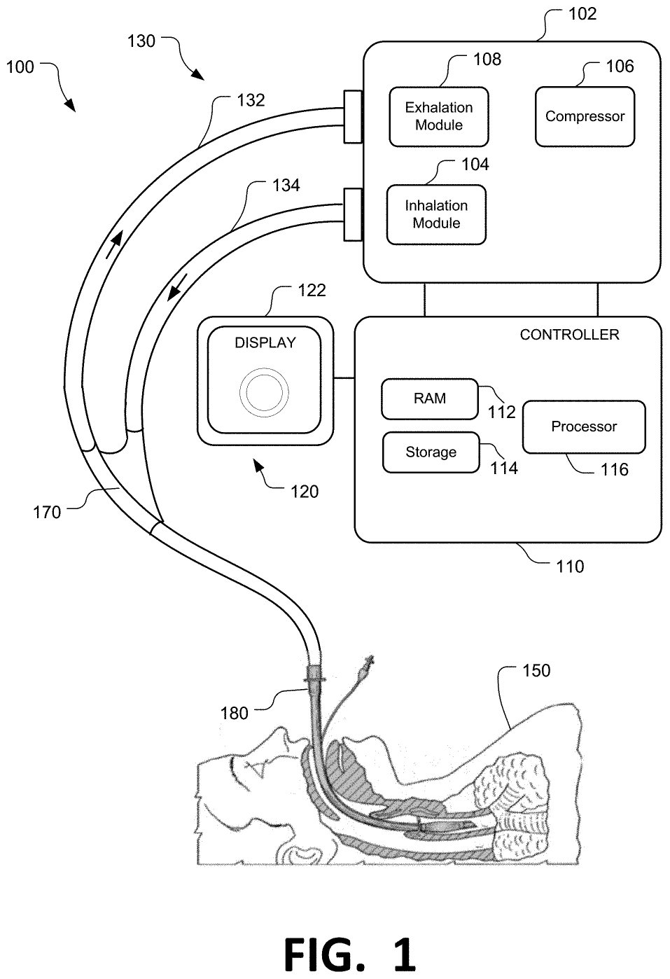

FIG. 1 is a diagram illustrating an example of a medical ventilator 100 connected to a patient 150. The ventilator 100 may provide positive pressure ventilation to the patient 150. Ventilator 100 includes a pneumatic system 102 (also referred to as a pressure generating system 102) for circulating breathing gases to and from patient 150 via the ventilation tubing (also called a breathing circuit) 130. The ventilation tubing 130 couples the patient 150 to the pneumatic system via a patient interface 180. The patient interface 180 may be invasive (e.g., endotracheal tube, as shown) or non-invasive (e.g., nasal or oral mask, nasal cannula). The ventilator 100 controls the flow of gases into the ventilation tubing 130 by controlling (adjusting, opening, or closing) an inhalation flow valve or blower which may be part of the inhalation module 104. Additionally, a humidifier may be placed along the ventilation tubing 130 to humidify the breathing gases being delivered to the patient 150. A pressure sensor and flow sensor may be located at or near the inhalation module 104 and/or the exhalation module 108 to measure flow and pressure.

The ventilation tubing 130 may be a two-limb circuit (shown, also called dual limb) or a one-limb circuit (also called single limb, with an inhalation side only). In a two-limb example, a wye-fitting 170, may be provided to couple the patient interface 180 to an inhalation limb 134 and an exhalation limb 132 of the ventilation tubing 130.

Pneumatic system 102 may have a variety of configurations. In the present example, system 102 includes an exhalation module 108 coupled with the exhalation limb 132 and an inhalation module 104 coupled with the inhalation limb 134. A compressor 106 or blower or other source(s) of pressurized gases (e.g., air, oxygen, and/or helium) is coupled with inhalation module 104 to provide breathing gas to the inhalation limb 134. The pneumatic system 102 may include a variety of other components, including mixing modules, valves, sensors, tubing, accumulators, filters, etc., which may be internal or external sensors to the ventilator (and may be communicatively coupled, or capable communicating, with the ventilator).

Controller 110 is operatively coupled with pneumatic system 102, signal measurement and acquisition systems, and a user interface 120. Controller 110 may include hardware memory 112, one or more processors 116, storage 114, and/or other components of the type found in command and control computing devices. In the depicted example, user interface 120 includes a display 122 that may be touch-sensitive and/or voice-activated, enabling the display 122 to serve both as an input and output device to enable a user to interact with the ventilator 100 (e.g., change ventilation settings, select operational modes, view monitored parameters, etc.).

The memory 112 includes non-transitory, computer-readable storage media that stores software that is executed by the processor 116 and which controls the operation of the ventilator 100. In an example, the memory 112 includes one or more solid-state storage devices such as flash memory chips. In an alternative example, the memory 112 may be mass storage connected to the processor 116 through a mass storage controller (not shown) and a communications bus (not shown). Although the description of computer-readable media contained herein refers to a solid-state storage, it should be appreciated by those skilled in the art that computer-readable storage media can be any available media that can be accessed by the processor 116. That is, computer-readable storage media includes non-transitory, volatile and non-volatile, removable and non-removable media implemented in any method or technology for storage of information such as computer-readable instructions, data structures, program modules or other data. For example, computer-readable storage media includes RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

As discussed briefly above, in clinical settings, a clinician may wish to temporarily adjust the oxygenation levels of a patient. For example, the patient may need a boost in oxygen levels, or the oxygen levels may need to be temporarily reduced. Temporarily adjusting the oxygenation levels of a ventilation system may be initiated by selecting a user interface element displayed on the graphic user interface of the ventilator. When the user interface element is selected, the oxygen concentration delivered from the ventilation system to the patient changes for a temporary period. Unlike prior systems and methods for adjusting oxygenation levels, the present technology can alter the oxygenation concentration in a customizable tiered and time-controlled manner. Each tier, or segment, in a cycle may have a different oxygen concentration and an associated timer. For instance, at the end of a first time segment (i.e., when the timer on the first segment has expired) the oxygen level is altered from the oxygen level of the first segment to the oxygen level of the second segment. At the end of the second time segment, the oxygen level is adjusted to the oxygen level of the third segment. This cycle may continue until the end of the final segment in the cycle is reached. The settings associated with the user interface element are selectable by a clinician. For example, the clinician can select the total time duration of the cycle for temporarily adjusting the oxygenation levels, and the clinician may also select the number of tiers in the cycle. Further, the time duration of each tier or segment is configurable, as well as the oxygen level of each segment.

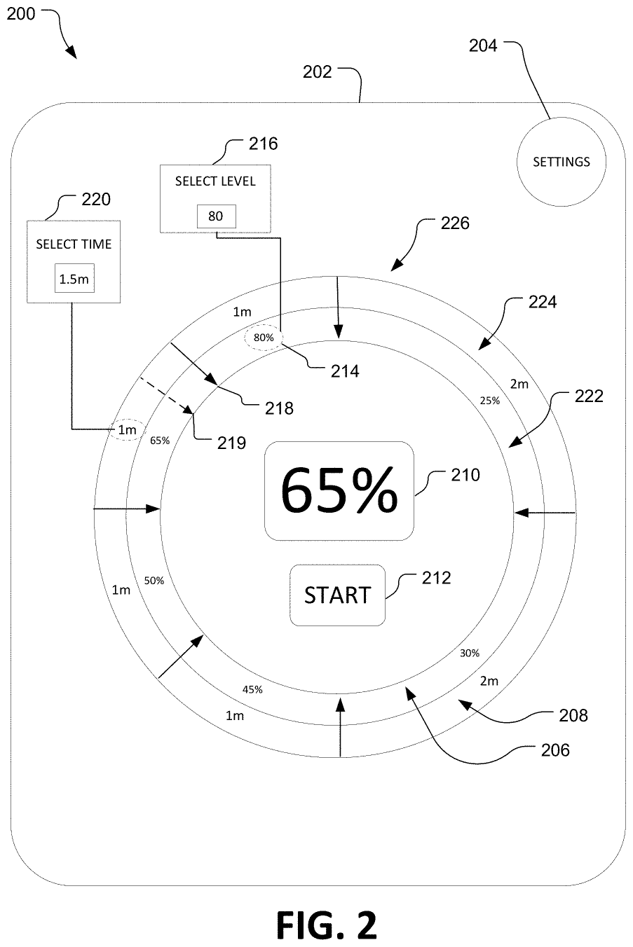

FIG. 2 depicts a display screen 200 coupled to a ventilation system. The display screen includes an example graphical user interface (GUI) 202 for presenting the status and operation of a temporary oxygenation adjustment function. Display screen 200 may be mounted to the ventilator or a separate screen, tablet, or computer that communicates with the ventilator. The GUI 202 may be an interactive display, e.g., a touch-sensitive screen or otherwise, and may provide various windows (i.e., visual areas) comprising elements for receiving user input and interface command operations and for displaying ventilatory information (e.g., ventilatory data such as pressure, volume, and flow waveforms, inspiratory time, PEEP, baseline levels, etc.), and control information (e.g., alerts, patient information, control parameters, modes, etc.). The elements may include controls, graphics, charts, tool bars, input fields, icons, etc. The display screen 200 may also include physical buttons or input elements, such as dials, wheels, switches, buttons, etc.

The GUI 202 displays a first bar 206 and a second bar 208 in concentric, adjacent rings. The first bar 206 includes a plurality of parameter segments 222 representing different tiers in the temporary oxygenation adjustment cycle. Each parameter segment 222 indicates an oxygenation level of a ventilation setting (e.g., 25%, 30%, 45%, 50%, 65%, 80%) corresponding to the tier represented by the respective parameter segment 224. The second bar 208 includes a plurality of time segments 224 for the different respective tiers. Each time segment 224 indicates a time duration (e.g., 1 minute, 2 minutes) for the respective tier. Each time segment 224 corresponds to a parameter segment of the first bar. The width of the time segments 224 and parameter segments 222 may be based on the time duration for the corresponding tier. For example, a time segment 224 with a longer time duration may be displayed as having a larger width than a time segment 224 having a shorter time duration. Because the parameter segments 222 may be aligned with the time segments 224, the parameter segments 222 share a width with their corresponding time segments 224.

The plurality of segments 222, 224 may be separated by dividers 218. In examples where each tier has the same time duration, the dividers 218 may automatically appear evenly spaced around the rings 226. The dividers 218 may be selectable and movable. In examples, the dividers may be selected and moved to configure the length of time of each of the plurality of time segments 224. For example, the divider 218 may be moved to a different position 219 to change the length of time of a time segment from 1 minute to 1.5 minutes. Additionally or alternatively, when a time indicator (e.g., 1.5 m) is selected, a select time window 220 may open on the GUI 202 and a user can input an amount of time for the selected time segment 224. The select time window 220 may include an input field and/or adjustment icons for increasing and/or decreasing the time interval. The select time window 220 may also be displayed upon selection of one of the dividers 218.

The level of the oxygenation setting for each of the plurality of parameter segments 222 is also configurable. When a parameter segment setting 214 is selected, a select oxygenation level window 216 may open on the GUI 202 and a user can input an oxygenation level for the selected parameter segment 222. The oxygenation level window 216 may include an input field and/or adjustment icons for increasing and/or decreasing the oxygenation level (e.g., fraction of inspired oxygen (FiO2) level) for that parameter segment.

The GUI 202 on the display 200 shown in FIG. 2 may further include a current oxygenation level indicator 210 showing the current oxygenation level that is being delivered to the patient. Thus, the clinician can easily identify, and see from afar, what is actually being delivered to the patient, without having to conduct any further investigation. When the first bar 206 and second bar 208 are displayed as concentric, adjacent rings 226, the current oxygenation level indicator 210 may be displayed within the middle of the rings 226. As the oxygenation level changes, the current oxygenation level indicator 210 changes to reflect the current oxygenation level. The GUI 202 may also include a selectable start/stop element 212 for controlling the starting and stopping of the temporary oxygenation adjustment function. Before the temporary oxygenation function is activated, while at time 0:00, the start/stop element 212 may display “START,” which when selected, will activate the selected mode (e.g., boost mood or reduce mode) and begin the temporary oxygenation adjustment cycle. During the temporary oxygenation adjustment cycle, the start/stop element 212 may change to display a “PAUSE” or “STOP” option.

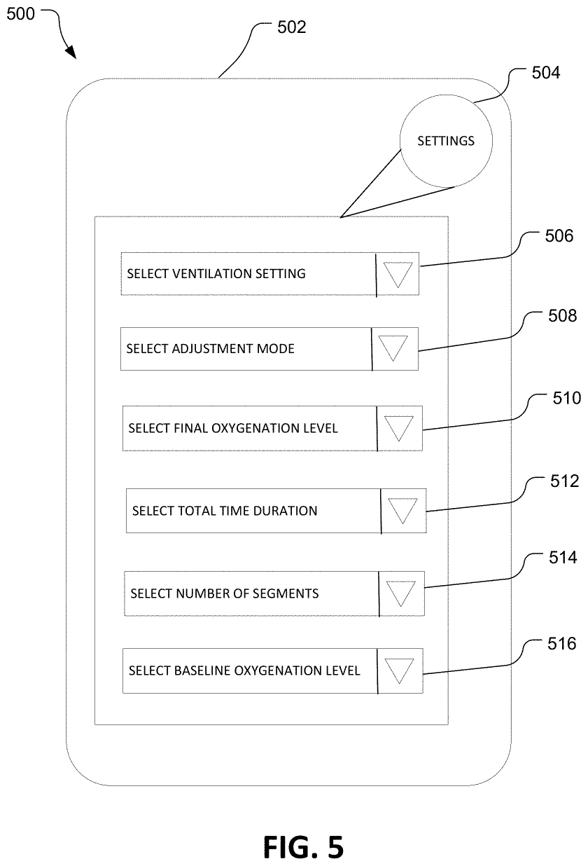

The GUI 202 may also include an oxygenation settings element 204 for inputting oxygenation settings to configure the temporary oxygenation adjustment function. For instance, upon selection of the oxygenation settings element 204, a settings interface may be displayed that provides additional or alternative options for changing the settings of the temporary oxygenation adjustment cycle. Such settings are described in more detail below with reference to FIG. 5.

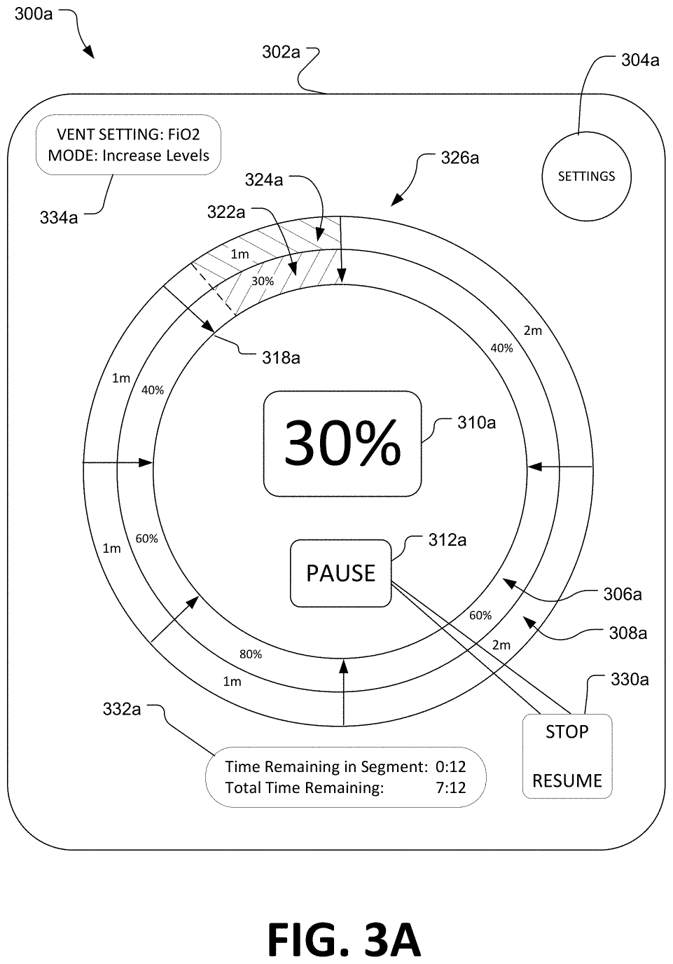

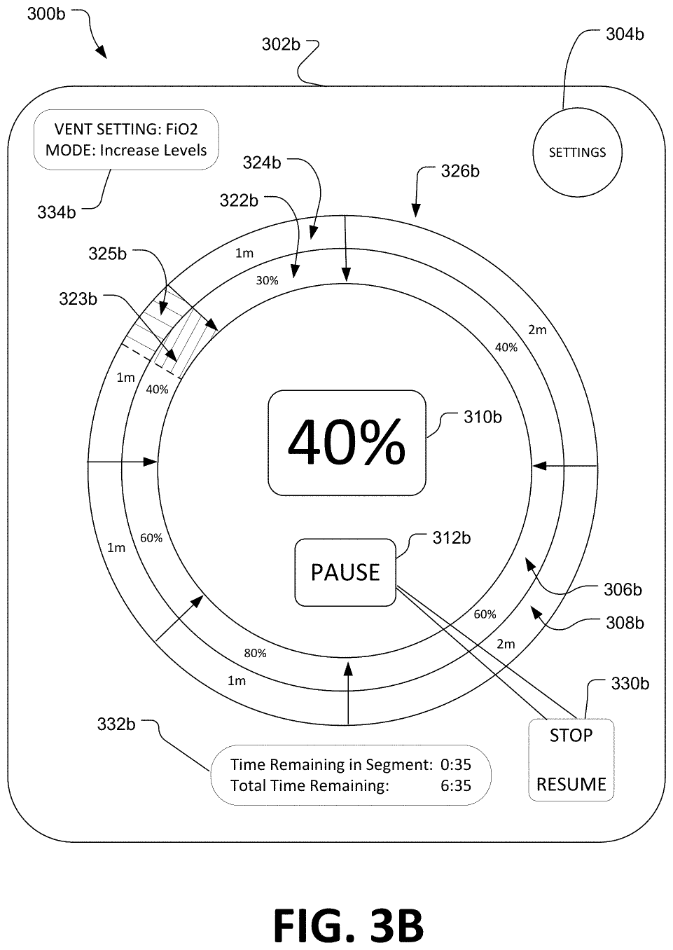

FIG. 3A is another example display screen 300a coupled to a ventilation system, depicting a graphical user interface 302a showing the status and operation of a temporary oxygenation adjustment function once a cycle has been activated. The GUI 302a includes an oxygenation settings element 304a and a current oxygenation level indicator 310a. The GUI 302a further includes a mode display window 334a indicating the current ventilation setting and the current mode. For instance, while the temporary adjustment cycle is described primarily described as changing an oxygen level (e.g., FiO2), the temporary adjustment cycle may be used for adjusting other types of ventilation settings as well, such as positive end-expiratory pressure (PEEP) or other types of ventilation settings. Accordingly, the mode display indicator or window 334a displays the particular ventilation setting that is being adjusted by the temporary adjustment cycle. The mode of the temporary adjustment cycle, such as increasing/boosting or decreasing the ventilation setting, may also be displayed in the mode display indicator or window 334a. In the example depicted in FIG. 3A, the current ventilation system cycle is temporarily increasing FiO2 levels.

The cycle in FIG. 3A is progressing through the first time segment 324a and corresponding parameter segment 322a. As the cycle progresses through the first segments 322a, 324a the segments may be highlighted or shaded to indicate the point at which the cycle is at within the segments 322a, 324a. The segments 322a, 324a may have the same highlighting or shading or different highlighting or shading. The current FiO2 level according to the first parameter segment is 30%, as displayed in the current oxygenation level indicator 310a. The FiO2 level will increase when the timer of the first segment 324a expires upon reaching the first segment divider 318a.

The GUI 302a may also include a time display window 332a. The time display window 332a may display both the time remaining in the current time segment, and the total time duration remaining in the cycle to reach the final oxygenation level of the ventilation setting. The GUI 302a may also include a selectable start/stop element 312a for controlling the starting and stopping of the temporary oxygenation adjustment function. As the cycle has already been activated, the start/stop element 312a may display “PAUSE,” which when selected, will pause the cycle. Once paused, further options to stop and end the cycle or to resume the cycle may be displayed on the GUI 302a in another window 330a.

FIG. 3B is another example display screen 300b coupled to a ventilation system, depicting a graphical user interface 302b showing the status and operation of the same temporary oxygenation adjustment function as in FIG. 3A at a different point in the cycle. In the example depicted in FIG. 3B, the cycle has completed the first time segment 324b and is currently progressing through the second time segment 325b. The second time segment 325b and second parameter segment 323b are highlighted to indicate at what point within the segments the cycle is at. Once a segment has been completed, the display may show completed segments greyed out. When all the segments have been completed and the total time remaining is 0:00, the current oxygenation level indicator 310b reflects the final oxygenation level. At this point, the oxygenation level may return to a default or baseline level. In examples, the oxygenation level may remain at the final oxygenation level for a set amount of time before returning to a default or baseline level.

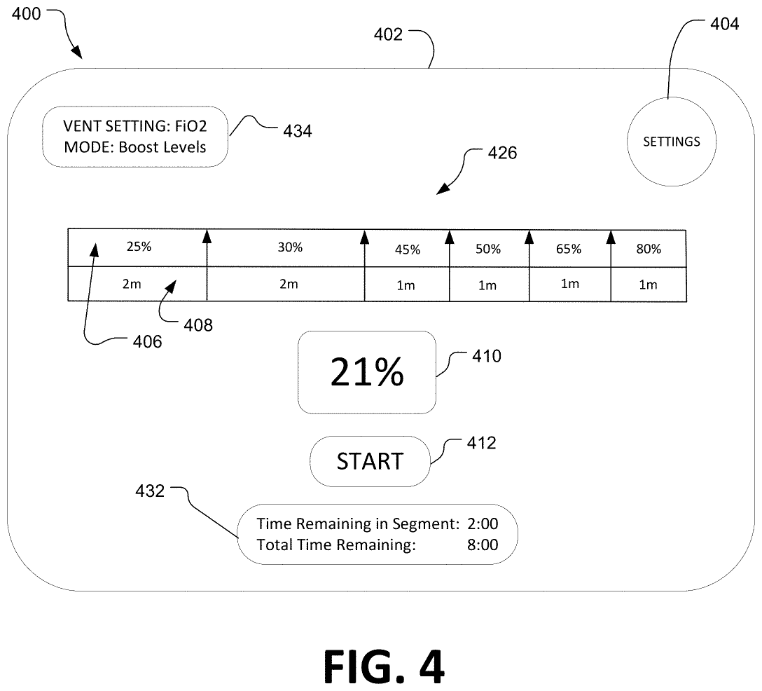

FIG. 4 is another example display screen 400 coupled to a ventilation system having an example graphical user interface (GUI) 402 for presenting the status and operation of a temporary oxygenation adjustment function. The GUI 402 may have an oxygenation settings element 404, a current oxygenation level indicator 410, a start/stop element 412, a mode display window 434, and a time display window 432. In the example shown in FIG. 4, the first bar 406, which contains a plurality of parameter segments, and the second bar 408, which contains a plurality of time segments, are displayed as linear adjacent bars 426. The parameter segments of the first bar 406 may be aligned with the time segments of the segment bar 408. In examples, there may be a setting that allows a user to select the configuration of the first bar and the second bar (e.g., concentric rings, linear bars, vertical bars). In the example depicted in FIG. 4, the first time segment and corresponding parameter segment appear on the left side of the GUI 402, and the cycle will proceed to the final oxygenation level at the far right side of the GUI 402 as time progresses.

FIG. 5 depicts a display screen 500 having an example graphical user interface (GUI) 502 configuring the settings of a temporary oxygenation adjustment function, wherein the display screen 500 is coupled to a ventilator. The GUI 500 may be displayed in response to receiving a selection of a settings element 504.

The GUI 502 may include an oxygenation settings element 504 for inputting settings to configure a temporary oxygenation adjustment function to operate on a ventilator system. A user, such as a clinician, may configure the oxygenation settings based on the patient's needs. When the oxygenation settings element 504 is selected, menus or options may be displayed. For example, multiple drop-down menus 506, 508, 510, 512, 514 may be displayed on the GUI 502. The drop-down menus 506, 508, 510, 512, 514 may include preset options to select from, as well as an option to customize an input. An oxygenation ventilation setting can be selected in drop-down menu 506, where the oxygenation ventilation setting may be, but is not limited to, a fraction of inspired oxygen (FiO2) or a positive end-expiratory pressure (PEEP). An oxygenation adjustment mode can be selected in drop-down menu 508, where the mode may be, but is not limited to, a boost mode or a reduce mode. Boost mode may be selected if a user or clinician determines a patient needs enhanced oxygenation levels. During a boost mode cycle, the oxygen levels will be increased over time until a final oxygenation level is reached. Reduce mode may be selected if a user or clinician determines a patient needs reduced oxygen levels. During a reduce mode cycle, the oxygen levels will be decreased over time until the final oxygenation level is reached. At the end of each cycle, the oxygen levels may return to a baseline level.

A final oxygenation level can be selected in the drop-down menu 510. In an example, when the ventilation setting is FiO2, the drop-down menu 510 may include options such as 100%, 90%, 80%, and 70%. The drop-down menu 510 may also include an option to customize the final oxygenation level, so the user can input a customized value for the final oxygenation level. A total time duration can be selected in drop-down menu 512. The total time duration is the amount of time it will take to reach the final oxygenation level (e.g., 6 minutes, 8 minutes, 10 minutes). A number of time segments can be selected from the drop-down menu 514. The number of time segments will correspond with the number of tiers to reach the final oxygenation level, where each segment or tier will correspond to an oxygenation level. In examples, a baseline oxygenation level may be programmed as part of the ventilator settings. If a user wishes to change the baseline, or first oxygenation level, the baseline oxygenation level, or first oxygenation level, can be selected from the drop-down menu 516 to select a new baseline oxygenation level. When an oxygenation cycle is complete, oxygen levels will return to the selected baseline oxygenation level. In other examples, additional drop-down menus may also be displayed on the GUI 502 to further configure the ventilation settings.

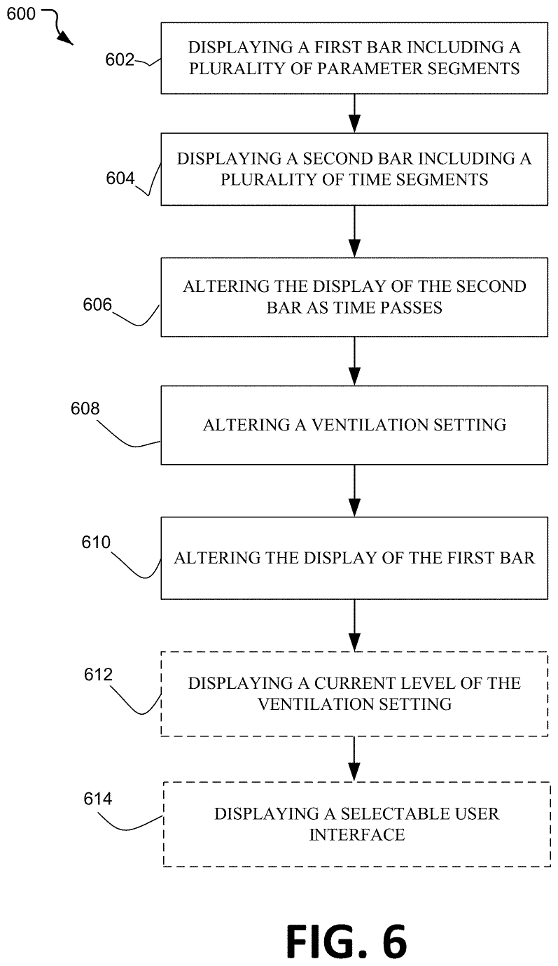

FIG. 6 depicts an example method 600 of controlling a temporary oxygenation adjustment function of a ventilation system using a graphical user interface (GUI). The method 600 may be performed by a ventilation system and include displaying user interface elements on a display screen of the ventilation system. Example suitable ventilation systems having a display screen are described above, for example, in FIGS. 1-5.

The method 600 includes, at operation 602, displaying, on a display screen of a ventilation system, a first bar including a plurality of parameter segments, where each parameter segment indicates a level of a ventilation setting, such as an oxygenation level, a PEEP level, or a level of another ventilation setting. A number corresponding to the level of the ventilation setting may be displayed within each parameter segment. The ventilation setting may be, but is not limited to, an oxygenation setting (e.g., FiO2 setting), or a PEEP setting. The method 600 further includes, at operation 604, displaying, on the display screen, a second bar including a plurality of time segments each time segment indicating a time duration of a corresponding parameter segment of the first bar. The length of each of the plurality of time segments is configurable by a user. Each of the plurality of time segments may be configured to be equal in time duration (e.g., 1 minute each), or different in time duration. Examples of the time duration for the plurality of time segments may include, but is not limited to, 0.5 minutes, 1 minute, 1.5 minutes, 2 minutes. As another example, each of the time durations may be less than 2 minutes, less than 5 minutes, and/or less than 10 minutes. The parameter segments of the first bar may be aligned with the time segments of the segment bar, and the first bar and the second bar may form concentric circles.

As time passes throughout the cycle, the display of the second bar having the plurality of time segments is altered, at operation 606, based on an amount of time passed. In examples, the display of the second bar may change by highlighting a portion of the time segment, where the amount of highlighting corresponds to the amount of time passed in the time segment. For example, if a time segment is 1 minute, and 30 seconds have passed, half of the time segment may be highlighted. When a time duration of a time segment expires, at operation 608, the ventilation system is altered to a level corresponding to a subsequent parameter setting. For instance, if the ventilation setting is FiO2, and a time segment with a corresponding parameter segment configured to 30% has just expired, and the subsequent parameter setting is 40%, upon the expiration of the time segment the ventilation system will alter the level to 40%. The ventilation system may alter the level to subsequent parameter setting immediately upon expiration of the time segment (i.e., immediately alter to 40%), or it may alter the level to the subsequent parameter setting more gradually throughout the subsequent time segment (i.e., over 1 minute gradually alter from 30% to 40%). For example, the level may be increased linearly over the time period for the segment.

The method 600 continues, at operation 610, by altering the display of the first bar to highlight the subsequent parameter setting. As the ventilation setting is altered in operation 608, the display of the first bar may be concurrently altered in operation 610. When the parameter segments of the first bar are aligned with the time segments of the segment bar, as time passes, altering the display of the second bar 606 and altering the display of the first bar 610 occurs concurrently such that the highlighting of the first bar aligns with the highlighting of the second bar.

The current level of the ventilation setting may be displayed on the display screen at operation 612. Where the first and second bars are displayed as concentric, adjacent rings, the current level of the ventilation setting may be displayed within the rings formed by the first bar and second bar, as depicted in FIGS. 3, 4A, 4B.

At operation 614, a selectable user interface element may also be displayed on the display screen. The selectable user interface element may control at least one of an activation of a boost mode, pausing of the boost mode, or stopping a boost mode. If the boost mode is paused, the ventilation system may be configured to automatically stop the boost mode after the expiration of a set amount of time, and return the level of the ventilation setting to a baseline level (e.g., the ventilation setting level that was used prior to initiation of the temporary adjustment cycle). The user interface may have additional elements for receiving input from a user. In examples, a selection of a ventilation level, total time duration, and number of time segments may be input from a user interface.

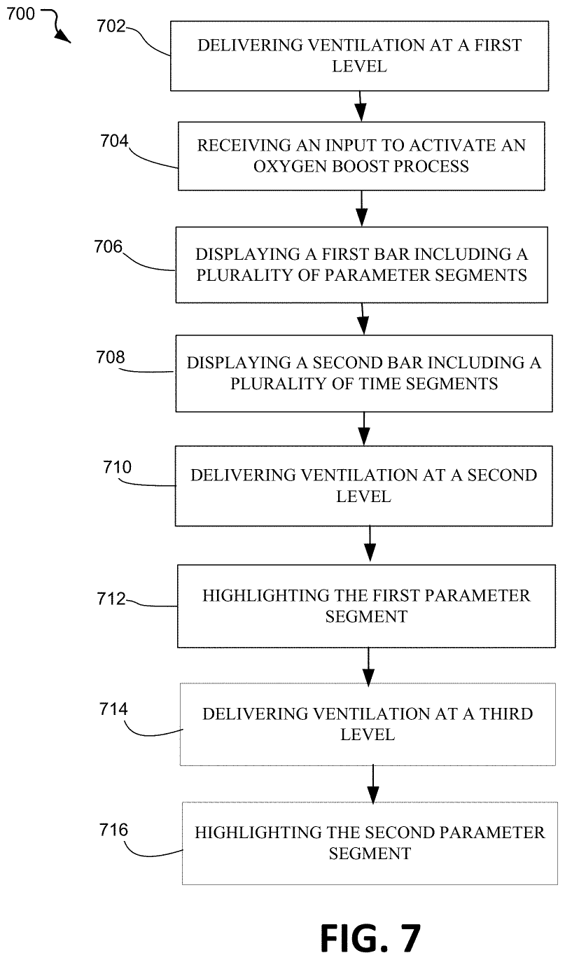

FIG. 7 depicts another example method 700 of controlling a temporary oxygenation adjustment function of a ventilation system using a graphical user interface. The method 700 includes, at operation 702, delivering ventilation at a first FiO2 level. The first FiO2 level may be a baseline level. At operation 704, an input may be received to activate an oxygen boost process. The input may be received from a clinician on a user interface. Method 700 continues, at operation 706, with displaying, on a display screen of a ventilation system, a first bar including a plurality of parameter segments. Each of the parameter segments may indicate a FiO2 level (e.g., oxygenation level). A second bar including a plurality of time segments aligned with corresponding parameter segments of the first bar is also displayed on the display screen at operation 708. Each time segment indicates a time duration of a corresponding parameter segment of the first bar. The first bar and the second bar may be arranged in concentric, adjacent rings.

At operation 710, in response to receiving the input to activate the oxygen boost process, ventilation is delivered at a second FiO2 level corresponding to a first parameter segment of the first bar. The second FiO2 level is greater than the first FiO2 level where a boost mode is selected. For example, the first FiO2 level may be 21%, and the second FiO2 level may be 25%. The method continues with highlighting the first parameter segment at operation 712. The amount of highlighting of the first parameter segment corresponds with the amount of time that has passed in the corresponding first time segment. Once the first time duration corresponding to the first time segment has expired, at operation 714, ventilation at a third FiO2 level is delivered corresponding to a second parameter segment of the second bar. At operation 716, the second parameter segment is highlighted accordingly. The third FiO2 level may be greater than the second FiO2 level.

In examples, upon expiration of a second time duration corresponding to a second time segment, ventilation may be delivered at a fourth FiO2 level corresponding to a third parameter segment. The fourth FiO2 level may be greater than the third FiO2 level to continue to boost FiO2 levels. In other examples, the fourth FiO2 level may be less than the third FiO2 level to reduce FiO2 levels. When ventilation is delivered at a level corresponding to the third parameter segment, the third parameter segment may be highlighted. The method 700 may continue for a plurality of time segments and parameter segments until a final level of a ventilation setting is reached. When the time duration of the final segment of the second bar expires, ventilation may be delivered at the first FiO2 level. If an input is received to stop the oxygen boost process, ventilation may also be delivered at the first FiO2 level.

In other examples, an input may be received to activate an oxygen reduce process. These example methods may proceed similarly to method 700, however, the second FiO2 level is less than the first FiO2 level, and the third FiO2 level is less than the second FiO2 level. In yet other examples, an input may be received to activate an oxygenation process that boosts and reduces oxygenation levels in a single cycle. For example, oxygenation levels may be increased over a set period of time, then the oxygenation level may be maintained at a final oxygenation level for a set period of time, and then the oxygenation levels may be reduced to a baseline oxygenation level over a set period of time.

Those skilled in the art will recognize that the methods and systems of the present disclosure may be implemented in many manners and as such are not to be limited by the foregoing aspects and examples. In other words, functional elements being performed by a single component, or multiple components, in various combinations of hardware and software or firmware, and individual functions, can be distributed among software applications at either the client or server level or both. In this regard, any number of the features of the different aspects described herein may be combined into single or multiple aspects, and alternate aspects having fewer than or more than all of the features herein described are possible.

Functionality may also be, in whole or in part, distributed among multiple components, in manners now known or to become known. Thus, a myriad of software/hardware/firmware combinations are possible in achieving the functions, features, interfaces, and preferences described herein. Moreover, the scope of the present disclosure covers conventionally known manners for carrying out the described features and functions and interfaces, and those variations and modifications that may be made to the hardware or software firmware components described herein as would be understood by those skilled in the art now and hereafter. In addition, some aspects of the present disclosure are described above with reference to block diagrams and/or operational illustrations of systems and methods according to aspects of this disclosure. The functions, operations, and/or acts noted in the blocks may occur out of the order that is shown in any respective flowchart. For example, two blocks shown in succession may in fact be executed or performed substantially concurrently or in reverse order, depending on the functionality and implementation involved.

Further, as used herein and in the claims, the phrase “at least one of element A, element B, or element C” is intended to convey any of: element A, element B, element C, elements A and B, elements A and C, elements B and C, and elements A, B, and C. In addition, one having skill in the art will understand the degree to which terms such as “about” or “substantially” convey in light of the measurement techniques utilized herein. To the extent such terms may not be clearly defined or understood by one having skill in the art, the term “about” shall mean plus or minus ten percent.

Numerous other changes may be made which will readily suggest themselves to those skilled in the art and which are encompassed in the spirit of the disclosure and as defined in the appended claims. While various aspects have been described for purposes of this disclosure, various changes and modifications may be made which are well within the scope of the disclosure. Numerous other changes may be made which will readily suggest themselves to those skilled in the art and which are encompassed in the spirit of the disclosure and as defined in the claims.

What is claimed is:

1. A method of controlling a ventilation system, the method comprising:
displaying, on a display screen of a ventilation system, a first bar including a plurality of parameter segments, each parameter segment indicating a level of a ventilation setting;
displaying, on the display screen, a second bar including a plurality of time segments, each time segment indicating a time duration of a corresponding parameter segment of the first bar;
as time passes, altering the display of the second bar based on an amount of time passing;
upon a time duration of a time segment expiring:
altering, by the ventilation system, the ventilation setting to a level corresponding to a subsequent parameter segment; and
altering the display of the first bar to highlight the subsequent parameter setting.

2. The method of claim 1, wherein the first bar and the second bar are displayed as concentric, adjacent rings.

3. The method of claim 1, further comprising displaying a current level of the ventilation setting within the rings formed by the first bar and the second bar.

4. The method of claim 1, further comprising displaying a selectable user interface element for controlling at least one of: activation of a boost mode, pausing of the boost mode, or stopping a boost mode.

5. The method of claim 1, wherein the ventilation setting is a fraction of inspired oxygen (FiO2) setting.

6. The method of claim 1, wherein the ventilation setting is a positive end-expiratory pressure (PEEP).

7. The method of claim 1, wherein each parameter segment displays a number corresponding to the level of the ventilation setting.

8. The method of claim 1, wherein the parameter segments of the first bar are aligned with the time segments of the second bar.

9. A ventilation system comprising:
a display screen; and
a controller including at least one processor and memory storing instructions that, when executed by the at least one processor cause the system to perform operations comprising:
displaying, on a display screen of a ventilation system, a first bar including a plurality of parameter segments, each parameter segment indicating a level of a ventilation setting;
displaying, on the display screen, a second bar including a plurality of time segments, each time segment indicating a time duration of a corresponding parameter segment of the first bar;
as time passes, altering the display of the first bar based on an amount of time passing;
upon a time duration of a time segment expiring:
altering, by the ventilation system, the ventilation setting to a level corresponding to a subsequent parameter segment; and
altering the display of the first bar to highlight the subsequent parameter setting.

10. The ventilation system of claim 9, wherein the display screen is attached to a ventilator housing.

11. The ventilation system of claim 9, wherein the ventilation setting is a fraction of inspired oxygen (FiO2) setting or a PEEP setting.

12. The ventilation system of claim 9, further comprising an oxygen valve, and wherein altering the ventilation setting includes adjusting the oxygen valve.

13. The ventilation system of claim 9, wherein the controller, when executed by the at least one processor causes the system to perform operations further comprising:
receiving, from a user interface, a selection of a final ventilation level, a selection of a total time duration, and a selection of a number of time segments.

14. The ventilation system of claim 13, wherein the duration of time of each of the plurality of time segments is selected by movement of a selector on a user interface.

15. The ventilation system of 13, wherein the level of the ventilation setting at each parameter segment is selected based on input received from a user interface.

16. A method for controlling a ventilator, the method comprising:
delivering ventilation at a first FiO2 level;
receiving an input to activate an oxygen boost process;
displaying, on a display screen of a ventilation system, a first bar including a plurality of parameter segments, each parameter segment indicating an FiO2 level;
displaying, on the display screen, a second bar including a plurality of time segments aligned with corresponding parameter segments of the first bar, each time segment indicating a time duration of a corresponding parameter segment of the first bar;
in response to receiving the input to activate the oxygen boost process:
delivering ventilation at second FiO2 level corresponding to a first parameter segment of the first bar, the second FiO2 level being greater than the first FiO2 level; and
highlighting the first parameter segment;
upon expiration of a first time duration corresponding to a first time segment of the second bar:
delivering ventilation at a third FiO2 level corresponding to a second parameter segment of the second bar; and
highlighting the second parameter segment.

17. The method of claim 16, wherein the third FiO2 level is less than the second FiO2 level and greater than the first FiO2 level.

18. The method of claim 16, wherein the third FiO2 level is greater than the second FiO2 level.

19. The method of claim 16, further comprising, upon expiration of a second time duration corresponding to a second time segment of the second bar:
delivering ventilation at a fourth FiO2 level corresponding to a third parameter segment of the second bar, the fourth FiO2 level being less than the third FiO2 level and greater than the first FiO2 level; and
highlighting the third parameter segment.

20. The method of claim 16, further comprising:
in response to one of receiving an input to stop the oxygen boost process or expiration of a time duration of a final segment of the second bar:
delivering ventilation at the first FiO2 level.
